(19) United States
(12) Patent Application Publication
(10) Pub. No.: US 2006/0190036 A1
(43) Pub. Date: Aug. 24, 2006

(54) Sealing plug for an opening in a wall of a vessel or hollow organ

(75) Inventors: Hans-Peter Wendel, Balingen (DE); Bernd Neumann, Rottenburg-Wurmlingen (DE)

Correspondence Address: KLARQUIST SPARKMAN, LLP, 121 SW SALMON STREET, SUITE 1600, PORTLAND, OR, 97204, US

(73) Assignee: Eberhard-Karls-Universitaet Teubingen

(21) Appl. No.: 11/343,510

(22) Filed: Jan. 30, 2006

Related U.S. Application Data
(63) Continuation of application No. PCT/EP04/08458, filed on Jul. 28, 2004.

(30) Foreign Application Priority Data
Jul. 30, 2003 (DE) DE 103 35 648

(51) Int. Cl. A61B 17/08

(52) U.S. Cl. 606/213

(57) ABSTRACT

The present invention relates to a sealing plug for an opening in a wall of a vessel or hollow organ of an animal or human body, in particular a blood vessel, to a device for placing such a sealing plug in such an opening, to a surgery kit for percutaneously sealing an opening in a wall of a vessel or hollow organ of an animal or human body, in particular a blood vessel, and to a method for percutaneously sealing of an opening in a wall of a vessel or hollow organ of an animal or human body.

### CROSS-REFERENCES TO RELATED APPLICATIONS

This application is a continuation of copending international patent application PCT/EP2004/008458 filed on Jul. 28, 2004 and designating the U.S., which was not published under PCT Article 21(2) in English, and claims priority of German patent application DE 103 35 648.7 filed on Jul. 30, 2003, which is incorporated herein by reference.

### BACKGROUND OF THE INVENTION

1. Field of the Invention

The present invention relates to a sealing plug for an opening in a wall of a vessel or hollow organ of an animal or human body, in particular a blood vessel, to a device for placing such a sealing plug in such an opening, to a surgery kit for percutaneously sealing an opening in a wall of a vessel or hollow organ of an animal or human body, in particular a blood vessel, and to a method for percutaneously sealing of an opening in a wall of a vessel or hollow organ of an animal or human body.

The present invention generally relates to devices and methods for percutaneously sealing openings in walls of vessels or hollow organs. The present invention in particular relates to the percutaneous sealing of arterial an venous puncture sites which usually are only accessible through the skin.

2. Related Prior Art

Many diagnostic and interventional vascular methods are performed transluminally, whereby a catheter is inserted into the vascular system at an appropriate access site and the same is guided through the vascular system to a target region, for doing so established techniques are used. This vascular access is e.g. created by the well-known Seldinger technique.

If the vascular access is no longer needed the inserted sluice tube has to be removed and the bleeding at the puncture site has to be stopped. Among the experts the sluice tube is also referred to as a sluice, access sluice, or introducer.

A percutaneous vascular access is for example needed to perform percutaneous transluminal angiologic interventions, for which the sluice tube that usually has a diameter of 5 to 8 French corresponding to 1.7 to 2.7 mm, mostly is inserted into the arteria femoralis. Examples for methods which are performed via such an access are angiography, angioplasty, laser ablation, stent placing, intravascular image recording, percutaneous transluminal coronary angioplasty etc. Other methods are also known for which a percutaneous access to hollow organs and lumens in the body are needed, for example laparoscopic methods, endoscopic methods and the like.

In order to avoid bleeding complications and to help the patient to get mobility rapidly it is important to stop the bleeding from e.g. the arterial vessel very quickly after the withdrawal of the sluice tube. This in particular causes problems with patients who received anti-coagulants, thrombocyte inhibitors or a lysis therapy.

A common method for stopping the bleeding is to tend the puncture site by means of manual pressure and a subsequent pressure bandage until the bleeding has stopped. It is a disadvantage with this method that it is very time consuming and a manual pressure of up to one hour is needed very often before hemostasis is ensured. A further problem of this method is that it is unpleasant for the patient because of the application of excessive pressure, and, furthermore, the corresponding blood vessel can be sealed completely which may result in ischemia and/or thrombosis.

To redress these problems U.S. Pat. No. 5,417,699 describes a suture apparatus having a long tube, at the distal end of the same needles and a corresponding suture material are arranged. The suture apparatus is inserted into the sluice tube which is still placed, until its distal end comes to recumbency in the lumen of the vessel which is to be sealed. Then the sluice tube is withdrawn to such an extent that it is liberated from the opening in the wall, which is to be sealed, and the distal end of the suture apparatus is released, whereupon the needles are straddling. The needles are now pushed outwardly through the wall which surrounds the opening to be sealed, then they are seized on their tips and again pulled into the sluice tube. During the pulling of the suture apparatus into the sluice tube the suture material that is attached to the distal end of the needles is now pulled through the wall and subsequently can be knotted.

The suture apparatus of the company Perclose Inc., Sunnyvale, Calif., which is distributed under the product name Prostar is technically very complicated and requires very experienced operating personnel. Furthermore, this system is expensive and complex in practice.

The oldest device for percutaneously sealing of openings in blood vessels is distributed under the product name VasoSeal® as a vascular hemostatic device from Datascope Corporation, Montvale, N.J., USA. The method is primarily based on the collagen induced thrombus generation. To this end a purified collagen plug is inserted, which induces the formation of a hemostatic cap directly above the puncture site.

In order to place the collagen plug properly the placed sluice tube has to be removed at first and it has to be replaced by a tube which has a relatively large diameter, through which the collagen plug is then put in place. To achieve hemostasis it is, however, often required to exert manual pressure for a certain time period which may cause problems associated therewith as already discussed above.

A method which is referred to as Angio-Seal® is based on mechanical forces which are applied according to the sandwich technique, as well as on collagen induced thrombus formation. The puncture site is blocked mechanically by means of an anchor in the inside of the artery, which guides and holds the collagen plug in the tissue. Also with this method the existing sluice tube has to be removed at first before a new sluice tube can be placed, through which the collagen plug is then brought to its proper position. The collagen plug is then pushed against the outer wall of the artery by a temper, whereby the temper is pushed on the collagen plug via a spring which simultaneously pulls from the inside the anchor via a suture material against the opening.

Both methods which were described last have the disadvantage that the existing sluice tube is firstly to be replaced by a new tube which may lead to big problems especially with strongly bleeding openings. Furthermore, the required manipulations are connected with a considerable extent of irritation which is also seen as a disadvantage.

In a method which is called Duett™ by Vascular Solutions, Minneapolis, Minn., USA, a balloon having an elliptical form is inserted into the lumen and should ensure a temporary sealing of the puncture site on the luminal side, however, it should not hinder the blood flow through the vessel excessively. Furthermore, a mixture of collagen and thrombin is applied, which supports the coagulation. This method has the disadvantage insofar, since there is the risk that the injected mixture gets into the blood vessel unintentionally, whereby on account of the balloon which is arranged in the lumen, there is the risk that the flow through the vessel is severely impeded. A further disadvantage is that the balloon has to be removed after the sealing of the larger opening, after doing so a further opening remains, however of a much smaller diameter.

All methods and devices for percutaneously sealing of openings in walls of vessels or hollow organs described so far have the disadvantage that they are either too time consuming or too expensive, or also not effective enough, since they are not able to counteract against the pressure which exists in the blood vessel to be sealed, having a value of 100 to 160 mmHg, to a sufficient extent.

### SUMMARY OF THE INVENTION

Against this background it is the object of the present invention to provide a sealing plug of the kind as mentioned at the outset, by means of which a punctured vessel or hollow organ can be sealed in a fast and simple manner, so that the bleeding is stopped immediately.

According to the invention this object is solved by a sealing plug for an opening in a wall of a vessel or hollow organ of an animal or human body, especially a blood vessel, having a sealing means insertable into the opening, and retaining means arranged on the sealing means, which anchor the sealing means in the wall.

The object underlying the present invention is totally solved in this way.

The sealing plug is placed with the sealing means in the opening, so that the same is essentially closed and no blood can leak through the opening. For retaining the sealing means in the opening, sealing means are provided, which anchor the sealing means in or to the wall.

The sealing means can be applied via a sluice tube which has been already placed, whereby the sealing plug expands after it is liberated from the sluice tube, folds out or releases the tension, and in doing so it anchors in or to the wall.

Against this background the object underlying the invention is also solved by a surgery kit for sealing an opening in a wall of a vessel or hollow organ of an animal or human body, especially a blood vessel, having a sleeve which can be inserted into a sluice tube which has already been placed in an opening, a sealing plug which is arranged in the sleeve, and a pusher to push the sealing plug out of the sleeve. Therefore, said surgery kit is a further subject of the invention.

In this connection it is preferred if the sealing plug is clamped into the sleeve under pretension of the retaining means, and the retaining means return elastically into their extended, tension-free basic form during the push-out of the sealing plug.

Against this background another subject of the present invention is a device for the application of such a sealing plug into an opening in the wall of a vessel or hollow organ of an animal or human body, especially a blood vessel, having a sleeve which can be inserted into a sluice tube which has already been placed in the opening, whereby the sleeve is adapted to receive a sealing plug, and a pusher to push the sealing plug out of the sleeve. It is preferred if the sleeve is adapted to receive a sealing plug which is under pretension.

Since the surgery kit is already delivered with a prepared sealing plug, the new device can so-to-say be “loaded” with the relevant sealing plugs on the spot.

Therefore, the invention is based on the idea to use a sealing plug which autonomously anchors in the opening and thereby already ensures the sealing of the blood vessel. In this manner it is not necessary to inhibit a residual bleeding over a longer time period by a manual tension or corresponding technical measures. The bleeding is rather stopped immediately when the sealing plug is anchored in the opening.

According to the invention in a method for percutaneously sealing a puncture in a vessel or a hollow organ a sealing plug is brought to the opening via a sluice tube which has been already placed, and will anchor there in the wall at the moment when the sluice tube has been withdrawn and thereby the sealing plug has been liberated.

The sealing plug is therefore responsible for the sealing of the opening as well as for the anchorage, complex manipulation as for example required for the use of a suture apparatus are no longer necessary.

It is generally preferred if the retaining means comprise blades which fit against the inside and outside of the wall after the sealing plug was applied into the opening.

This has the advantage that the blades which fit against the inside as well as the outside of the wall of the vessel or hollow organ, on the one hand they prevent the sealing plug to be pushed out of the opening, on the other hand they also prevent the sealing plug to be pulled into the lumen. Furthermore, the blades which fit against the inside and the outside of the wall, in addition to the sealing means which already seals a large part of the diameter of the opening, ensure an immediate stop of the bleeding. Especially these blades enable that the sealing plug can be applied via the placed sluice tube, what means in reverse that the diameter of the sealing means is smaller than the diameter of the opening in the wall caused by the sluice tube. The opening in the wall is reduced after the withdrawal of the sluice tube, however it cannot be predicted whether after the withdrawal the opening will already be reduced to such an extent, that the sealing means by itself will be sufficient for an immediate and total sealing. The blades, therefore, contribute to the secure sealing immediately after the application of the new sealing plug.

In this connection it is preferred if the sealing means comprises an upper and a lower sealing surface, whereby on each sealing surface blades are arranged, whereby the sealing surfaces are preferably shaped as a polygon and a blade is arranged on each edge of the sealing surface.

Basically it is possible to form the sealing means as a cylindrical body. However, if the sealing surfaces and thus normally also the sealing means are formed as a polygon, the blades can be attached directly to the edges of the sealing surfaces. This has the advantage that during the application of the sealing plug via the sluice tube the blades can be folded-up or folded-down, respectively, and only unfold when the sluice tube is withdrawn. With a straight edge this “folding” is easier to realize from a geometrical view and has a lower tension than with a circular or curved edge.

In this connection it is preferred if the blades extent in their extended, tension-free basic form basically perpendicular to the longitudinal axis of the sealing means.

This measure has the advantage that the blades automatically return into their basic form after their liberation from the sluice tube or from a sleeve which transports them through the sluice tube, therefore it is not necessary to fold down the blades actively, for example by means of guidance cables, suture material etc., after the sealing plug has been placed in the correct position in the opening. Therefore, this measure also serves to apply the sealing plug in a simple manner, whereby the sealing plug is, so-to-say, automatically brought into the correct position in the opening due to the blades which fit against the inside and the outside of the wall.

It is furthermore preferred, if the blades are provided with anchoring means which clasp into the wall or the surrounding tissue, whereby the anchoring means are preferably formed on the blades as barbed hooks or projections.

Also this measure ensures a fast and proper sealing of the opening. The sealing means is tightly remained in the opening by the anchoring means so that it is not necessary after the application of the sealing plug to exert manual or other tension over a longer time period. In other words, even for a vessel with a high internal pressure or a high flow rate the plug rapidly and securely anchors into the wall of the vessel or hollow organ.

Generally it is preferred if the blades are attached to the sealing means via a shape-elastic joint, whereby the blades preferably consist of a shape-elastic material.

This measure is advantageous in a constructive view and, therefore, in view of the costs, since the shape-elasticity by which the blades are transferred from their folded-up position into their folded-down position, is realized by the material of the blades or of their joints at the sealing means. Also because of this measure, further manipulations or the use of springs etc. are not necessary.

Furthermore, it is preferred if the sealing means and the blades are integrally formed of a shape-elastic material, i.e. formed as a single piece, respectively.

This measure has a constructive advantage, since the sealing plug can, for example, be produced by ejection-molding technology, whereby in the extended tension-free basic form the blades stand away from the sealing means laterally. The blades are then folded upwardly and downwardly, and, under pretension of the retaining means, i.e. of the blades, the sealing means can either be inserted directly into an existing sluice tube or into a corresponding device via which the sealing means are transported to the opening through the sluice tube.

The material is preferably selected from the group consisting of: elastomer, material comprising a shape memory effect, nitinol, polymer comprising shape memory effect.

If elastomers are used it is made use of their caoutchouc-like, elastic behavior which ensures that even after a strong deformation a return into the initial form takes place.

When a material is used, which comprises a shape memory effect it is taken advantage of the capability of these materials to readopt the initial form after a plastic deformation at the moment they are warmed beyond a characteristic temperature. A subject which is made of such a material “remembers” its initial form, that is why this effect is also referred to as a memory effect. Nitinol is a nickel titanium alloy which comprises such a shape memory effect.

Also polymers are known which comprise a shape memory effect. In this connection it is a particular advantage that the material can be absorbable in the body.

Absorbable polymers which comprise a shape memory effect are manifoldly described in the art; see for example U.S. Pat. No. 6,160,084, US 2003/0055198 A1 or EP 1 000 958 A1.

If the sealing plug consists of a shape memory material it can be produced in its tension-free basic form, so that the blades push against the inside and the outside of the wall of the vessel or hollow organ. The sealing plug is then warmed up and brought into its temporary form in which it can be applied through the sluice tube. This temporary form is fixed by cooling below the so-called transition temperature. If the sealing plug is then again warmed, the initial form is re-established. Polymer systems are available which have a transition temperature that is in the range of the ambient or body temperature. Such polymer systems are described in above-mentioned property rights and are, for example, distributed by the company mnomoScience GmbH, Pauwelsstraβe 19, D-52974 Aachen, Germany.

A sealing plug which is formed of such a polymer system is, if applicable, maintained under a low pretension in the sleeve of the surgery kit according to the invention, where the sealing plug is in its temporary form and is held at a temperature which is considerably below the body temperature. After the application of the sealing plug the latter comes into contact with body fluid and warms to a temperature beyond the transition temperature. Then the sealing plug readopts its stable, tension-free basic form, what means that the blades fold down and anchor the sealing means in the opening against the wall.

It goes without saying that also with a material which has a shape memory, the folding-down of the blades can largely or exclusively be effected by the elasticity or by a folding ability.

Since the material is absorbable it is gradually degraded during the following days and weeks, whereby the cells of the vessel wall automatically grow into the gap which results from the absorption. After the sealing plug is completely degraded, the vessel wall has been regenerated in the area of the puncture site to such an extend that the opening remains securely sealed.

In this connection it is preferred if the material is covered with medically effective substances or contains the same, for example, the material is covered with collagen and/or an antibiotic is mixed into the material.

In this manner on the one hand the healing process of the wall can be promoted, whereas on the other hand by the antibiotic, for example, the incidence of inflammations can be avoided.

It is generally preferred if the sealing plug is produced of a polymer which is absorbable in the body and which comprises a shape memory effect.

This measure has the advantage that the sealing plug can be produced on a low cost basis, and it can be applied rapidly and easily due to the above-mentioned reasons thus ensuring a secure and rapid sealing of the opening in the wall of the vessel or hollow organ.

According to an advantageous further development the retaining means of the sealing plug, which extend along the longitudinal axis of the vessel or hollow organ after the application has taken place, are longer than the retaining means which extend perpendicular to the longitudinal axis of the hollow organ. In this connection it is further preferred if the retaining means which extend perpendicular to the longitudinal axis of the hollow organ, are, in their tension-free basic form, arch-shaped, so that after the application of the sealing plug in the opening said retaining means fit against the inside and outside of the wall of an approximately cylindrically shaped blood vessel.

These measures have the advantage that the plug is adapted to the cylindrical form of the blood vessel or hollow organ, so that an injury of the wall thereof is prevented and, simultaneously, a sealed fit of the plug is ensured.

For the surgery kit it is then preferred if the pusher protrudes upwardly beyond a stop which is provided on the sleeve, to such an extent that the pusher when inserted into the sleeve pushes the sealing plug downwardly and out of the sleeve just that far that the lower retaining means are liberated from the sleeve and release their tension.

This measure has the advantage that the stop has merely to be inserted into the sleeve as far as necessary to, for the time being, partially push the sealing plug out of the sleeve. It is therefore not required to take care of the exact insertion course of the pusher.

Since the proximal end of the sleeve is disposed in the lumen of the vessel or hollow organ to be sealed, the lower end of the sealing plug now extends into the lumen where the retaining means, i.e. especially the blades, release their tension and form a kind of disc. If the sluice tube and therewith the sleeve is now withdrawn from the opening the lower retaining means come into contact with the inside of the wall of the vessel or hollow organ. If the withdrawal of the sluice is continued the sleeve is then removed from the rest of the sealing plug and also the upper retaining means are liberated and can release their tension correspondingly.

According to the invention a sealing plug is placed in the opening which is provided in a wall, in such a manner, that it is firstly brought with its lower end into the lumen of a blood vessel or hollow organ, whereupon the lower end is liberated and unfolds its retaining means. During the withdrawal of the sluice and/or the sleeve the lower retaining means retain the sealing plug in the opening in such a way that it is completely liberated from the sluice tube or the sleeve, respectively.

In this connection it is especially preferred if an arrest is provided between the sleeve and the pusher.

This measure has the advantage that when the sleeve is inserted into the sluice tube the pusher is not actuated unintentionally, but the arrest has to be removed or to be overcome at first. As an arrest, for example, an overridable catch, a spring, but also a stop can be used, which has to be removed at first.

In this connection it is further preferred if a lug is provided on the outside of the sleeve, the position of which on the sleeve is adjusted in such a manner that the sleeve is insertable into the sluice tube just as far that the lower end of the sleeve is approximately in flush with the outlet of the sluice tube.

This measure has the advantage that when the sleeve is inserted into the sluice tube no special care has to be taken on how far the sleeve can be inserted. It is therefore not required to check the position of the proximal end of the sleeve.

After the required interventions by the use of the disposed sluice tube are finished, it is only necessary to insert the sleeve until the hit of the stop to ensure the correct position of the sealing plug for its further application. Afterwards the pusher is pushed and after a short retention period during which the lower retaining means release their tension, the sluice tube is removed in conjunction with the sleeve disposed therein.

In this manner, a very easy application of the sealing plug is enabled, whereby the new method in general can be carried out rapidly and easily, and for the operating personnel no extensive briefing for the new system is required.

In this connection it is preferred if the lug is a flange which is arranged on the outside of the sleeve in a movable and adjustable manner.

This has the advantage that the surgery kit can be adapted to the length of the sluice tube which is used in each case. It is therefore not required to provide different surgery kits for sluice tubes having different lengths.

On the other side it is preferred if the sleeve comprises an outer diameter that is adapted to the inner diameter of sluice tubes.

This has the advantage that for respective sluice tubes, the different diameters thereof are standardized, one respective surgery kit can be provided and a sealing plug with an optimal diameter is applied by the respective sluice tube.

A further subject matter of the invention is a method for percutaneously sealing of openings in walls of vessels or hollow organs of a human or animal body, especially of arterial or venous puncture sites, comprising the steps: (a) inserting a sluice tube into the opening; (b) providing a sealing plug in a sleeve; (c) inserting the sleeve with the sealing plug provided therein into the sluice tube; (d) pushing the sealing plug out of the sleeve which was inserted into the sluice tube, by means of a pusher, so that the sealing plug is placed in the opening and the same is essentially sealed.

Further advantages result from the description and the enclosed drawing.

It goes without saying that the features named above and those still to be explained below can be used not only in the respectively specified combination, but also in other combinations or on their own without departing from the scope of the present invention.

The drawing shows an embodiment which is explained in further detail in the following description.

### BRIEF DESCRIPTION OF THE DRAWINGS

FIG. 1 shows in a perspective side view of a blood vessel with a disposed sluice tube;

FIG. 2 shows a surgery kit consisting of a sleeve, a pusher, and sealing plug which was inserted into the sluice tube;

FIG. 3 shows a sluice tube in its temporary form which can be used in a surgery kit as shown in FIG. 2, during the folding-down of the retaining means and in their tension-free basic form;

FIG. 4 shows a blade of the sealing plug as shown in FIG. 3 comprising a sharpened projection;

FIG. 5 shows a blade of the sealing plug as shown in FIG. 3 comprising a blanked-out barbed hook; and

FIG. 6 shows the process of the application of the sealing plug by the use of the surgery kit shown in FIG. 2.

### DESCRIPTION OF PREFERRED EMBODIMENTS

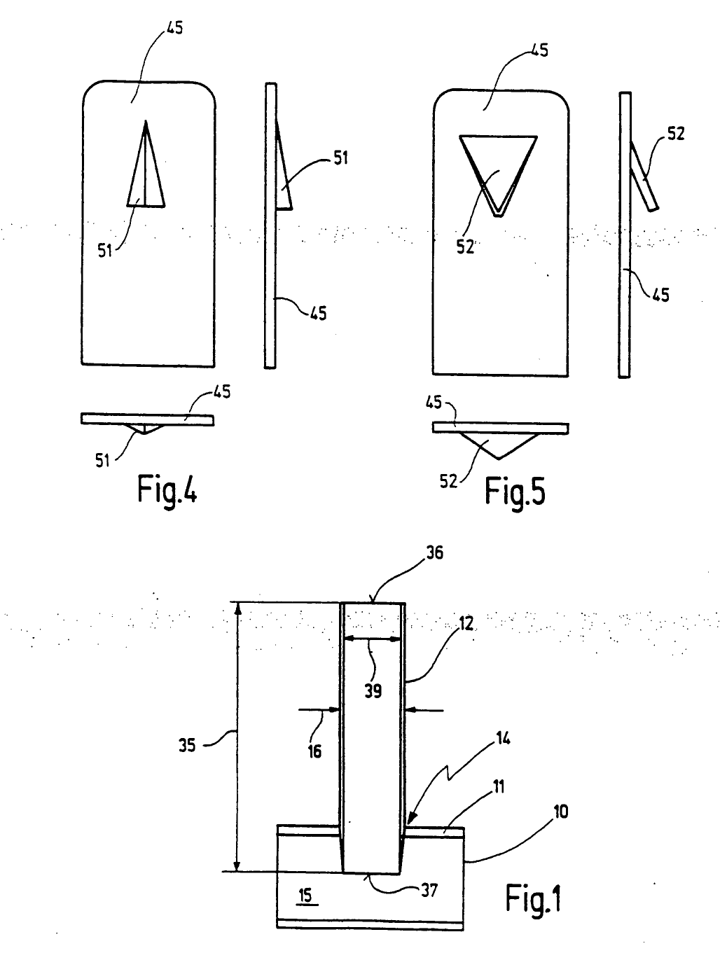

FIG. 1 shows a schematic side view of a segment of a blood vessel 10, in the wall 11 thereof a sluice tube 12 was inserted, so that an opening 14 is formed via which the lumen 15 of the blood vessel 10 is accessable. The sluice tube 12 has an outer diameter 16 which approximately defines the size of the opening 14.

A sluice tube 12 which is, for example, needed for radiologic or cardiologic interventions, is used for inserting a catheter into the lumen 15.

After the withdrawal of the sluice tube 12 the opening 14 remains which may cause fulminant bleedings in case of no further treatments, especially when the blood vessel 10 is e.g. the arteria femoralis.

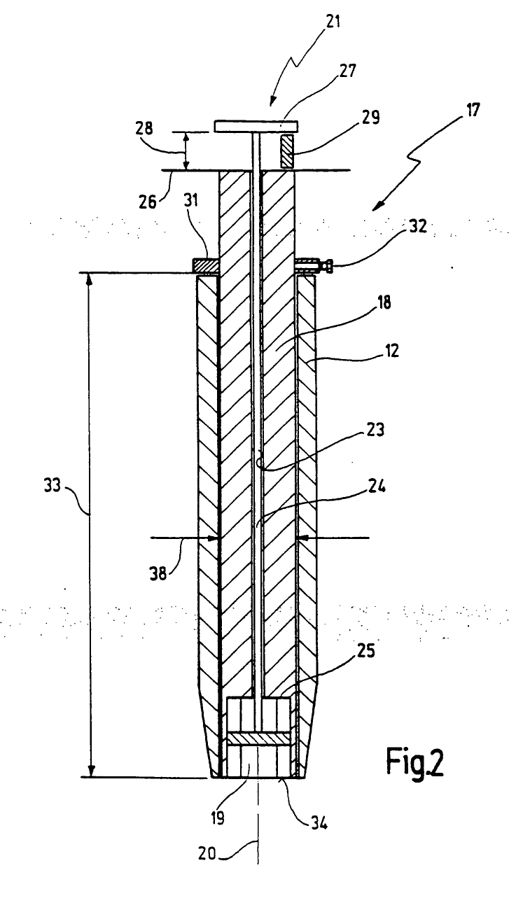

In order to seal the opening a surgery kit 17 is used as this is shown in a schematic section and in a side view in FIG. 2.

The surgery kit 17 includes a sleeve 18 in which a sealing plug 19 is disposed, the longitudinal direction thereof which is indicated at 20 is in coincidence with a pusher 21 which is guided by its rod 24 in a bore 23 which is provided in the sleeve 18.

A room 25 is provided in the sleeve 18, in which the sealing plug 19 is disposed.

The sleeve 18 is provided at its distal end with a stop 26 which in the simplest case can be a diagonally arranged plastic sheet. The rod 24 comprises at its upper end a head 27 which, in the basic position of the surgery kit 17 as shown in FIG. 2, has a distance 28 from the stop 26. This distance 28 is much smaller than the extension of the sealing plug 19 along its longitudinal direction 20, so that in the case of a total insertion of the rod 24 into the sleeve 18 only half of the sealing plug 19 is pushed out of the room 25.

In order to prevent an unintentional actuation of the pusher 21 an arrest which is indicated at 29 is provided, which prevents a motion of the head 27 towards the stop 26. The arrest 29 can be a stop which has to be removed at first before the pusher 21 can be actuated. It is also possible that the arrest 29 comprises an overridable catch or a spring so that the pusher 21 can be inserted by applying a stronger pressure which does, however, not occur unintentionally.

On the outside of the sleeve 18 a ring-shaped flange 31 is provided which can be moved lengthwise along the sleeve and can be adjusted in its respective position by a knurled screw 32 so that a distance 33 to the lower end 34 of the sleeve 18 is established.

The flange 31 is adjusted in such a manner that the distance 33 corresponds to the length 35 of the sluice tube 12; see FIG. 1.

The surgery kit 17 as shown in FIG. 2 was now inserted into the sluice tube 12 wherefore it is plugged into its entry aperture 36 (FIG. 1) as far as the flange 31 attaches the entry aperture. The lower end 34 of the sleeve 18 is now approximately in flush with the exit aperture 37 of the sluice tube 12.

In order to insert the surgery kit 17 into the sluice tube 12 it is required that the sleeve 18 comprises an outer diameter 38 which is slightly smaller than the inner diameter of the sluice tube 12, which is indicated in FIG. 1 at 39.

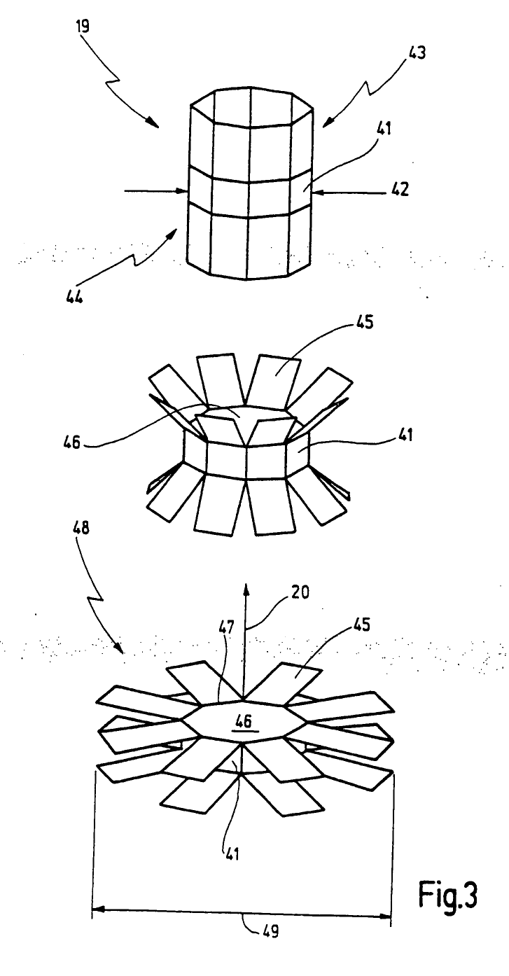

The sealing plug which is shown in FIG. 2 is shown in FIG. 3 in more detail.

First of all the sealing plug 19 comprises a sealing means 41, the outer diameter thereof which is indicated at 42 is adapted in such a manner that it can be placed in the room 25 of the surgery kit 17, where it is held under slight pretension, where appropriate.

Upper and lower retaining means 43 and 44, respectively, are attached to the upper side and the lower side of the sealing means 41, each of said retaining means are formed as blades 45.

The illustration in the middle of FIG. 3 shows the blades 45 declined outwards to a certain degree, so that between them an upper sealing surface 46 is visible. This upper sealing surface 46 as well as a lower sealing surface which cannot be seen in FIG. 3 are each formed as a polygon, whereby a blade 45 is arranged on each edge of the polygon.

The sealing plug as described so far is integrally produced, i.e. produced as a single piece, of a polymer having a shape memory effect, which is absorbable in the body, by means of injection molding technology.

In the lower part of FIG. 3 the sealing plug 19 is shown in its tension-free basic form 48, in which the blades 45 extend approximately perpendicular to the longitudinal axis 20 of the sealing means 41. The blades 45 have a transverse extension which is indicated at 49, which is much larger than the diameter 14 determined by the outer diameter 16 of the sluice tube 12.

In the upper part of FIG. 3 the sealing plug 19 is shown in its temporary form and below its transition temperature it remains in this form in a relatively tension-free state. However, if the temperature of the sealing plug 19 is increased beyond the transition temperature which is above the room temperature however below the body temperature, the blades 45 fold down and again pass into the tension-free basic form as shown in the lower part of FIG. 3, via the form as shown in the middle of FIG. 3. This folding-down of the blades 45 can also be caused only due to the elasticity of the material.

The material of the sealing plug 19 is a polymer system as it is, for example, supplied by the company mnemoScience. Furthermore, the sealing plug 19 can be covered with an appropriate medical substance, for example with collagen, to improve the healing process. Furthermore, a medically effective substance, for example an antibiotics, can be mixed to the polymer to prevent inflammations.

It is to be mentioned that the material is absorbable whereby the sealing plug disintegrates in the body in a well-known manner, and due to the ongoing healing process the sealing means is replaced by endogenic tissue.

When the sealing means 41 is applied into the opening 14 it seals the opening 14 almost completely. However, since its outer diameter 42 is smaller than the outer diameter 16 of the sluice tube 12, the blades 45 ensure not only the stability of the sealing means in the opening 14, but they also form a kind of disc above and below the wall 11 and thus seal any gaps which may exist between the wall 11 and the sealing means 41.

In this connection the blades 45 are attached in a sealing and retaining manner against the outside as well as the inside of the wall 11 of the blood vessel 10, whereby this attachment can, if appropriate, take place under a slight pretension. In this manner the blades 45 retain the sealing means 41 in position and ensure the additionally needed sealing.

To ensure a particularly secure stability of the sealing plug 19 in the opening 14 the blades 45 are provided with anchoring means which are shown in FIGS. 4 and 5.

In FIG. 4 a projection 51 which is provided on the blade 45 is sharpened, whereas in FIG. 5 a barbed hook 52 is blanked out of the blade 45. The projection 51 or the barbed hook 52, respectively, ensure the anchorage of the folded-down blades in the wall 11 or the surrounding tissue, so that the sealing plug 19 is securely fixed in the wall 11. The geometry of the blades 45 can also be anti-traumatically rounded, star-shaped, pointed or can have another suited shape.

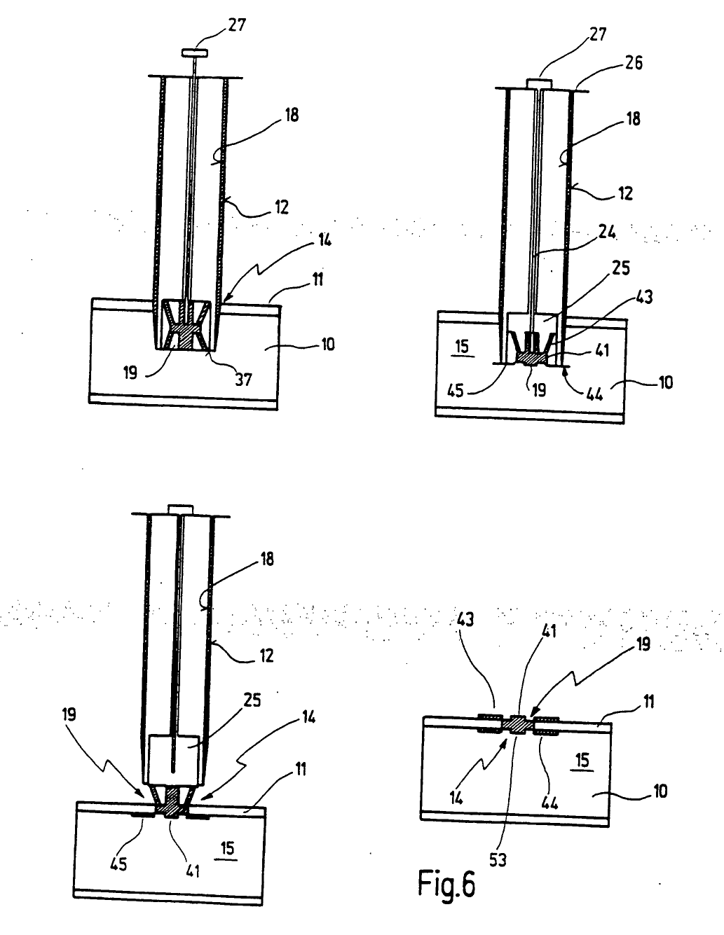

The way of the application of the sealing plug 19 into the opening 14 is now explained with reference to FIG. 6.

In the top left-hand corner of FIG. 6 the situation is shown in which the sluice tube 12 is still disposed in the opening 14, however, the sleeve 18 has already been inserted into the sluice tube. The sealing plug is now directly located at the exit aperture 37 of the sluice tube 12.

Now the head 27 is pushed downwardly towards the stop 26, whereby the rod 24 pushes approximately half the sealing plug 19 out of the room 25 and into the lumen 15 of the blood vessel 10. The lower retaining means 44, i.e. the blades 25, can now release their tension and return into their tension-free basic form in which they extend approximately perpendicular to the sealing means 41 and the sleeve 18 stands on the blades 45.

This transition of the blades 45 into the tension-free basic form takes place as a result of the elasticity of the material or because the sealing plug 19 has been warmed beyond its transition temperature due to the contact with the body fluid.

In case the sealing plug 19 was not made of a material having a shape memory effect but of an elastomer, the latter is held in the room 25 under a stronger pretension, however the blades 45 now also return into their basic form on account of their elasticity. This status is shown in the top left-hand corner of FIG. 6.

The sluice tube 12 is now withdrawn from the opening 14, so that the sealing means 41 reaches its position in the opening 14 and the blades 45 of the lower retaining means 44 then attach themselves against the inside of the wall 11. The opening 14 is then already sealed since the blood pressure in the lumen 15 pushes the disc formed by the blades 45 outwardly against the wall 11, and the disc in combination with the sealing plug 41, especially its lower sealing surface 53, seals the opening 14. When the sluice tube 12 is withdrawn further the sleeve 18 will be completely removed from the sealing plug 19, so that also the upper retaining means 43 will be liberated from the room 25 and return into their tension-free basic form, as this has already been described in connection with the lower retaining means 44.

The further withdrawal of the sluice tube 12 is shown in the lower left-hand corner of FIG. 6, whereas in the lower right-hand corner of FIG. 6 the sealing plug 19 is shown when anchored in the opening 14, which is now sealing the opening 14 completely. The outer retaining means 43 push against the outside of the wall 11 and thus prevent the sealing plug 19 to be carried away by a strong current in the lumen 15.

The sealing plug 19 as described so far has been tested in view of its suitability for securely sealing a puncture site. The tested sealing plug 19 was integrally made of nitinol and comprised the shape as shown in FIG. 3.

In a model consisting of tube materials, through which liquid was streamed, a segment of an aorta of pig having a length of 20 cm was inserted, and in this model internal pressures of up to 200 mmHg could have been tested. The sealing plug 19 was then inserted into the aorta in such a manner that it takes up its form and position as shown in the lower right-hand corner of FIG. 6.

It was demonstrated that the sealing plug 19 was not only securely stabilized in the opening 14 even at internal pressures of 200 mmHg and high flow rates, but also that the sealing plug 19 ensures a complete sealing of the opening 14. Directly after the application of the sealing plug 19 the opening 14 was sealed and remained sealed even during a longer assay run.

Therefore, what is claimed, is:

1. Sealing plug for an opening in a wall of a vessel or hollow organ of an animal or human body, especially a blood vessel, comprising a sealing means insertable into the opening, which seals the same, and retaining means arranged on the sealing means, which anchor the sealing means in the wall.

2. Sealing plug of claim 1, wherein the retaining means comprise blades which fit against the inside and the outside of the wall after the sealing plug was applied into the opening.

3. Sealing plug of claim 2, wherein the sealing means comprises an upper and a lower sealing surface, whereby blades are arranged on each sealing surface.

4. Sealing plug of claim 3, wherein the sealing surfaces are formed as a polygon and a blade is arranged on each edge of a sealing surface.

5. Sealing plug of claim 2, wherein the blades extend in their extended, tension-free basic form approximately perpendicular to the longitudinal axis of the sealing means.

6. Sealing plug of claim 2, wherein the blades are provided with anchoring means which clasp into the wall or the surrounding tissue.

7. Sealing plug of claim 6, wherein the anchoring means on the blades are shaped as barbed hooks or projections.

8. Sealing plug of claim 2, wherein the blades are attached to the sealing means via a shape-elastic joint.

9. Sealing plug of claim 2, wherein the blades consist of a shape-elastic material.

10. Sealing plug of claim 2, wherein the sealing means and the blades are integrally made of a shape-elastic material.

11. Sealing plug of claim 9, wherein the material is selected from the group consisting of: elastomer, material having a shape memory effect, nitinol, polymer having a shape memory effect.

12. Sealing plug of claim 9, wherein the material is absorbable in the body.

13. Sealing plug of claim 9, wherein the material is covered with medically effective substances and/or contains the same, for example the material is covered with collagen and/or an antibiotic is mixed into the material.

14. Sealing plug of claim 1, wherein it is produced by injection molding technology of a polymer which is absorbable in the body, and which comprises a shape memory effect.

15. Sealing plug of claim 1, wherein the retaining means which extend along the longitudinal axis of the vessel or hollow organ after the application has taken place, are longer than the retaining means which extend perpendicular to the longitudinal axis of the vessel or hollow organ.

16. Sealing plug of claim 15, wherein the retaining means which extend perpendicular to the longitudinal axis of the vessel or hollow organ, are, in their tension-free basic form, arch-shaped, so that after the application of the sealing plug in the opening said retaining means fit against the inside and outside of the wall of an approximately cylindrically shaped vessel or hollow organ.

17. Surgery kit for sealing an opening in a wall of a vessel or hollow organ of an animal or human body, especially a blood vessel, comprising a sleeve which can be inserted into a sluice tube placed in the opening, a sealing plug which is arranged in the sleeve, and a pusher to push the sealing plug out of the sleeve.

18. Surgery kit of claim 17, wherein the sealing plug is the sealing plug of claim 1.

19. Surgery kit of claim 18, wherein the sealing plug is clamped into the sleeve under pretension of the retaining means, and the retaining means release their tension and move back elastically into their extended tension-free basic form, after the sealing plug was pushed out.

20. Surgery kit of any of claim 17, wherein the pusher protrudes upwardly beyond a stop which is provided on the sleeve, to such an extent that the pusher when inserted into the sleeve pushes the sealing plug downwardly and out of the sleeve just that far that the lower retaining means are liberated from the sleeve and release their tension.

21. Surgery kit of claim 20, wherein an arrest is provided between the sleeve and the pusher.

22. Surgery kit of claim 17, wherein a lug is provided on the outside of the sleeve, whereby the position of the lug on the sleeve is adapted to the length of the sluice tube in such a way that the sleeve is insertable into the sluice tube just as far that the lower end of the sleeve is approximately in flush with the exit aperture of the sluice tube.

23. Surgery kit of claim 22, wherein the lug is a flange which is arranged on the outside of the sleeve in a movable and adjustable manner.

24. Surgery kit of claim 17, wherein the sleeve has an outer diameter that is adapted to the inner diameter of sluice tube.

25. Device for the application of a sealing plug into an opening in a wall of a vessel or hollow organ of an animal or human body, especially a blood vessel, comprising a sleeve for inserting into a sluice tube which is disposed in the opening, whereby the sleeve is constructed to receive a sealing plug, and a pusher for pushing the sealing plug out of the sleeve.

26. Device of claim 25, wherein the sleeve is the sleeve (18) of the surgery kit of claim 17.

27. Device of claim 25, wherein the pusher is the pusher of the surgery kit of claim 17.

28. Method for percutaneously sealing of openings in walls of vessels or hollow organs of a human or an animal body, especially of arterial or venous puncture sites, comprising the steps:
(a) inserting a sluice tube into the opening;
(b) providing a sealing plug in a sleeve;
(c) inserting the sleeve with the sealing plug provided therein, into the sluice tube;
(d) pushing the sealing plug out of the sleeve which was inserted into the sluice tube, by means of a pusher, so that the sealing plug is placed in the opening in such a way that the opening is essentially sealed.

29. Method of claim 28, wherein the sealing plug is the sealing plug of claim 1.

30. Method of claim 28, wherein the sluice tube is the sluice tube of claim 17.

31. Method of claim 28, wherein the sleeve is the sleeve of claim 17.

32. Method of claim 28, wherein the pusher is the pusher of claim 17.
